(19) United States
(12) Patent Application Publication
(10) Pub. No.: US 2024/0139550 A1
(43) Pub. Date: May 2, 2024

(54) METHOD OF CONSECUTIVE ADHERENCE AND DURATION SIGNALS FOR BONE HEALING

(71) Applicant: Bioventus LLC, Durham, NC (US)

(72) Inventors: Debra Ann ARRINGTON, Bartlett, TN (US); Christopher Herbert CHIAVARIO, Memphis, TN (US); Kim Patterson KELLY, Cary, NC (US); Alessandra PAVESIO, Chapel Hill, NC (US); Tawana Faye WESTER, Raleigh, NC (US); Neill Malcolm POUNDER, Cary, NC (US)

(21) Appl. No.: 18/384,082

(22) Filed: Oct. 26, 2023

Related U.S. Application Data
(60) Provisional application No. 63/419,905, filed on Oct. 27, 2022.

(51) Int. Cl. A61N 7/00

(52) U.S. Cl. CPC A61N 7/00; A61N 2007/0013

(57) ABSTRACT

Described herein is a novel method of treating bone fractures using low-intensity pulsed ultrasound.

### TECHNICAL FIELD

Described herein is a novel method of treating bone fractures using low-intensity pulsed ultrasound.

### BACKGROUND OF THE INVENTION

Bone fractures are a common injury for individuals worldwide. Post-injury rehabilitation can be a long process with weakened mobility and functioning. Patients may also experience delayed unions or non-unions respectively. An estimated 10% of individuals suffer from improper healing. (Einhorn et al., 2014) The normal healing process begins with an inflammatory phase post-injury that initiates formation of matrices and clotting. This is followed by a fibrovascular phase to reform damaged blood vessels and restore nutrient supply. Revascularization is coupled to a phase of bone formation. Stem cells needed for bone growth may then be recruited and differentiated into either osteoblasts or chondrocytes or other bone formation cells to finish restoring bone structure at the fracture site. (Bahney et al., 2019).

One approach to treating bone fractures is the use of therapeutic ultrasound to accelerate and aid in proper healing. Average treatments consist of low-intensity pulsed ultrasound in an optimal frequency range for an optimal time period. Ultrasound results in the passage of periodic mechanical sound waves through tissues that may aid in all biological stages of bone healing. The rebuilding of blood vessels and initial bone growth is also promoted by ultrasound treatment through modulation of growth-related and differentiation signaling. Finally, ultrasound is believed to aid in the bone remodeling phase in a similar fashion to mechanical forces or physical loading. (Jiang et al., 2018).

Poor patient compliance presents additional hurdles for recovery and healing, especially where a patient is required to self-administer therapy. However, direct feedback compliance for patients self-administering ultrasound therapy has been found have a significant impact on patient compliance. (Pounder et al., 2016).

### INCORPORATION BY REFERENCE

All publications, patents, and patent applications mentioned in this specification are herein incorporated by reference with regard to their background teachings to the same extent as if each individual publication, patent, or patent application was specifically and individually indicated to be incorporated by reference.

### SUMMARY OF THE INVENTION

In one embodiment described herein is a method of treating bone fracture in a patient with comorbidities, in need thereof, comprising administering to the patient 30 continuous treatments of a low-intensity pulsed ultrasound to the bone. In one aspect of the method the comorbidities comprise smoking, diabetes, obesity, or cardiovascular disease. In another aspect of the method, the bone comprises short bones and long bones. In another aspect of the method, the short bone comprises a scaphoid. In another aspect of the method, the long bone comprises a tibia.

In another aspect, the method further comprises tracking the patient's treatment. In another aspect, tracking comprises using a compliance calendar, a goal progress tracker or a continuity tracker.

In another aspect, the method further comprises administering to the patient about 60 to about 120 continuous treatments. In another aspect, the method further comprises administering to the patient about 60 continuous treatments. In another aspect, the method further comprises administering to the patient about 90 continuous treatments. In another aspect, the method further comprises administering to the patient about 120 continuous treatments.

In another aspect, the method further comprises healing the bone to complete healing. In another aspect, the method further comprises healing that is clinically significant.

Another embodiment described herein is a method of reducing the incidence of non-union fracture of a bone in a patient in need thereof comprising: a) administering 30 continuous treatments of a low-intensity pulsed ultrasound to a bone; b) tracking the patient's treatment; and c) healing the bone to complete healing. In another aspect, the bone comprises scaphoid or tibia bone. In another aspect, the tracking comprises using a compliance calendar, a goal progress tracker or a continuity tracker. In another aspect, the method further comprises administration of additional 30 continuous treatments.

### BRIEF DESCRIPTION OF THE DRAWINGS

The features of the present disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1: describes clinician survey results demonstrating average number of days of low-intensity pulsed ultrasound use for treating bone fractures.

FIG. 2: describes tracking tools a patient may use to track treatments and ensure adherence.

FIG. 3: describes the non-union odds ratio (ITT) for patients using EXOGEN® to a matched cohort.

### DETAILED DESCRIPTION OF THE DISCLOSURE

The following description and examples illustrate embodiments of the present disclosure in detail.

It is to be understood that the present disclosure is not limited to the particular embodiments described herein and as such may vary. Those of skill in the art will recognize that there are variations and modifications of the present disclosure, which are encompassed within its scope.

All terms are intended to be understood as they would be understood by a person skilled in the art. Unless defined otherwise, all technical and scientific terms used herein have the same meaning as commonly understood by one of ordinary skill in the art to which the disclosure pertains.

The section headings used herein are for organizational purposes only and are not to be construed as limiting the subject matter described.

Although various features of the disclosure may be described in the context of a single embodiment, the features may also be provided separately or in any suitable combination. Conversely, although the present disclosure may be described herein in the context of separate embodiments for clarity, the present disclosure may also be implemented in a single embodiment. The following definitions supplement those in the art and are directed to the current application and are not to be imputed to any related or unrelated case, e.g., to any commonly owned patent or application. Although any methods and materials similar or equivalent to those described herein may be used in the practice for testing of the present disclosure, the preferred materials and methods are described herein. Accordingly, the terminology used herein is for the purpose of describing particular embodiments only, and is not intended to be limiting.

In this application, the use of the singular includes the plural unless specifically stated otherwise. It must be noted that, as used in the specification, the singular forms “a,” “an” and “the” include plural referents unless the context clearly dictates otherwise.

In this application, the use of “or” means “and/or” unless stated otherwise. The terms “and/or” and “any combination thereof” and their grammatical equivalents as used herein, may be used interchangeably. These terms may convey that any combination is specifically contemplated. Solely for illustrative purposes, the following phrases “A, B, and/or C” or “A, B, C, or any combination thereof” may mean “A individually; B individually; C individually; A and B; B and C; A and C; and A, B, and C.” The term “or” may be used conjunctively or disjunctively, unless the context specifically refers to a disjunctive use.

Furthermore, use of the term “including” as well as other forms, such as “include,” “includes,” and “included,” is not limiting.

Reference in the specification to “some embodiments,” “some aspects,” “an embodiment,” “an aspect,” “another aspect”, “one embodiment,” “one aspect” or “other embodiments” or “other aspects” means that a particular feature, structure, or characteristic described in connection with the embodiments is included in at least some embodiments, but not necessarily all embodiments, of the present disclosures.

As used in this specification and the claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification may be implemented with respect to any method or composition of the disclosure, and vice versa. Furthermore, compositions of the present disclosure may be used to achieve methods of the present disclosure.

The term “about” or “approximately” means within an acceptable error range for the particular value as determined by one of ordinary skill in the art, which will depend in part on how the value is measured or determined, i.e., the limitations of the measurement system. For example, “about” may mean within 1 or more than 1 standard deviation, per the practice in the art. Alternatively, “about” may mean a range of up to 20%, up to 10%, up to 5%, or up to 1% of a given value. In another example, the amount “about 10” includes 10 and any amounts from 9 to 11. In yet another example, the term “about” in relation to a reference numerical value may also include a range of values plus or minus 10%, 9%, 8%, 7%, 6%, 5%, 4%, 3%, 2%, or 1% from that value. Alternatively, particularly with respect to biological systems or processes, the term “about” may mean within an order of magnitude, preferably within 5-fold, and more preferably within 2-fold, of a value. Where particular values are described in the application and claims, unless otherwise stated the term “about” meaning within an acceptable error range for the particular value should be assumed.

The term “comorbidity” as used herein refers to the simultaneous presence of two or more medical conditions and/or diseases in a patient. In the present disclosure, a comorbidity may refer to a disease or medical condition in addition to a bone fracture. Any comorbidity is contemplated herein, and includes for example, cardiovascular disease, cirrhosis, chronic obstructive pulmonary disease (COPD), osteoporosis, diabetes, cancer, renal insufficiency, obesity, or arthritis. Furthermore, comorbidity may also include concomitant medication use, including for example, anticoagulant use, anticonvulsant use, benzodiazepine use, bisphosphonate use, cardiac mediation use, diuretic use, hormone replacement therapy use, insulin use, anti-diabetic meds (non-insulin) use, non-steroidal anti-inflammatory (NSAID) use, opioid use, oral contraceptive use, steroid use, smoking cessation product use, proton pump inhibitor drug use, SSRI anti-depressant drug use, or thyroid drug use.

The term “composition” as used herein is intended to encompass a product comprising specific ingredients in specified amounts, as well as any product which results, directly or indirectly, from combination of the specified ingredients in the specified amounts. By “pharmaceutically acceptable” it is meant the carrier, diluent or excipient must be compatible with the other ingredients of the formulation, including the vectors described herein, and not deleterious to the recipient thereof. A “pharmaceutically acceptable carrier” is any carrier which is relatively non-toxic and innocuous to a patient at concentrations consistent with effective activity of the active ingredient so that any side effects ascribable to the carrier do not vitiate the beneficial effects of the active ingredient.

As used herein the term “continuous treatment” refers to consecutive, daily treatments using low-intensity pulsed ultrasound at specific settings.

As used herein the term “non-union fracture” refers to a non-healing fracture. The most agreed-upon definition of non-union is a fracture that persists without signs of healing for three months. Non-union fractures may also refer to non-healed fractures with no signs of healing after clinical evaluation when examined by a physician and using radiographs taken consecutively 45 to 90 days apart.

“Patient” or “subject” as used herein refers to a mammalian subject diagnosed with or suspected of having a bone fracture. In some embodiments, the term “patient” refers to a mammalian subject with a higher than average likelihood of developing a non-union bone fracture. Exemplary patients may be humans, apes, dogs, pigs, cattle, cats, horses, goats, sheep, rodents and other mammalians that may benefit from the therapies disclosed herein. Exemplary human patients may be male and/or female. “Patient in need thereof” or “subject in need thereof” is referred to herein as a patient diagnosed with or suspected of having a disease or disorder, for instance, but not restricted to rheumatoid arthritis.

“Administering” is referred to herein as providing one or more compositions or therapies as described herein to a patient or a subject. By way of example and not limitation, composition administration, e.g., injection, may be performed by intravenous (i.v.) injection, sub-cutaneous (s.c.) injection, intradermal (i.d.) injection, intraperitoneal (i.p.) injection, or intramuscular (i.m.) injection. One or more such routes may be employed. Parenteral administration may be, for example, by bolus injection or by gradual perfusion over time. Alternatively, or concurrently, administration may be by the oral route. Additionally, administration may also be by surgical deposition of a bolus or pellet of cells, or positioning of a medical device. In an embodiment, a composition of the present disclosure may comprise a gel used with a low-intensity pulsed ultrasound device, in an amount that is effective to treat, or heal a bone fracture. Administering may further refer to administration of a therapy of the present disclosure which may comprise administering low-intensity ultrasound, in an amount that is effective to treat, repair or promote healing of a bone fracture in a patient in need thereof.

As used herein, the term “treatment”, “treating”, or its grammatical equivalents refers to obtaining a desired pharmacologic and/or physiologic effect. In some embodiments, the effect is therapeutic, i.e., the effect partially or completely cures a disease and/or condition. In some embodiments, the term “treating” may include inhibiting or “preventing” a disease or a condition, including treating bone fracture. In some embodiments, the term “treating” may include “repairing” or “healing” a bone fracture.

As used herein, a “treatment interval” refers to a treatment cycle, for example, a course of administration of a therapeutic agent or therapy that may be repeated, e.g., on a regular schedule. In some embodiments, a dosage regimen may have one or more periods of no administration of the therapeutic agent in between treatment intervals.

The term “therapeutically effective amount”, therapeutic amount”, or its grammatical equivalents refers to an amount effective, at dosages or settings, and for periods of time necessary, to achieve a desired therapeutic result. The therapeutically effective amount may vary according to factors such as the disease state, age, sex, and weight of the individual, and the ability of a composition described herein to elicit a desired response in one or more subjects. The precise amount of the compositions or therapy of the present disclosure to be administered may be determined by a physician with consideration of individual differences in age, weight, tumor size, extent of inflammation, infection or metastasis, and condition of the patient (subject). For example, a therapeutically effective amount of low-intensity pulsed ultrasound maybe that amount, that specific settings, recommended by a medical device manufacturer or as prescribed by a physician over a treatment period.

As used herein the term “treatment period” refers to a prescribed course of a specific medical treatment, for example, low-intensity pulsed ultrasound treatments for a bone fracture, administered at specific settings over a period of time, for example a period of days. Thus, a treatment period may comprise administration of a low-intensity pulsed ultrasound for a treatment period of, for example, 1-120 days, 1-90 days, 1-60 days, 1-30 days or 1-15 days.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

The present disclosure describes a novel method of treating a bone fracture using low-intensity ultrasound. The method includes tracking tools to assist in patient adherence and results in clinically significant healing in a shorter time than the current standard of care.

Although there are currently successful treatments for bone fractures, post-injury treatment duration remains one challenge for positive patient outcomes. Elderly patients or patients with comorbidities are even further susceptible to complications in healing and reoccurrence as compared to those without. Use of ultrasound to therapeutically treat and evaluate bone injuries is known. However, the success of ultrasound treatment depends on patient compliance with treatment regimes. (Harrison et al., 2021) Shorter treatment duration has been shown to increase patient compliance as well as produce positive economic benefits due to shorter healing times and decreased risk of improper healing. (Sklar et al., 2008) Thus, one embodiment described herein is a method of treating a bone fracture in a patient in need thereof using low-intensity pulsed ultrasound.

Ultrasound delivery systems generally use pulsed radio-frequency waves (in the MHz range) to treat bone fractures. These systems take advantage of the piezoelectric nature of bones. When ultrasound is applied to the fracture bone, the ultrasound is converted to an electric current in the bone to promote healing by small deflections within the bone. Such systems are well known, and include, for example, those described in U.S. Pat. Nos. 5,003,965, 5,186,162, 5,556,372, and the EXOGEN® Ultrasound System, all of which are incorporated herein by reference with regard to such general teachings. However, other ultrasound application devices may be used in the methods described herein. In one aspect, the ultrasound is a low-intensity pulsed ultrasound.

Ultrasound therapy may be used to treat fractures of any bone type including long and short bones of the arms and legs, short bones of the wrists and ankles, flat bones such as shoulder bleads, breast one and skull bones, and irregular bones such as the vertebrae. In one aspect, the bone comprises humerus bones, metatarsal bones and malleolus bones. In one aspect, the bone comprises phalange bones including the distal phalanx of the thumb, proximal phalanx of the thumb, or any of the distal, middle or proximal phalange bones. In another aspect the bones comprise metacarpal bones. In another aspect the bones comprise the carpal bones including the ulna and radius bone, trapezoid, trapezium, capitate, scaphoid, lunate, triquetrum, hamate pisiform, or hamulus bones. In another aspect, the bone comprises any of the leg bones, including the femur, patella, tibia or fibula. In one aspect, the bone comprises a scaphoid or tibia bone.

Ultrasound therapy treatment may include administration of the ultrasound therapy at specific settings and for defined treatment periods. Treatment settings include specified intensities, frequencies and wavelengths. For example, the EXOGEN® Ultrasound Bone Healing System (Model Number 71034400) device uses a standard setting of 30 mW/cm 2 power density, 1.5 MHz signal, and 1 kHz, 20% duty cycle on time. Thus, in one aspect, the treatment may comprise daily treatment at a standard setting of for a low-intensity pulsed ultrasound device.

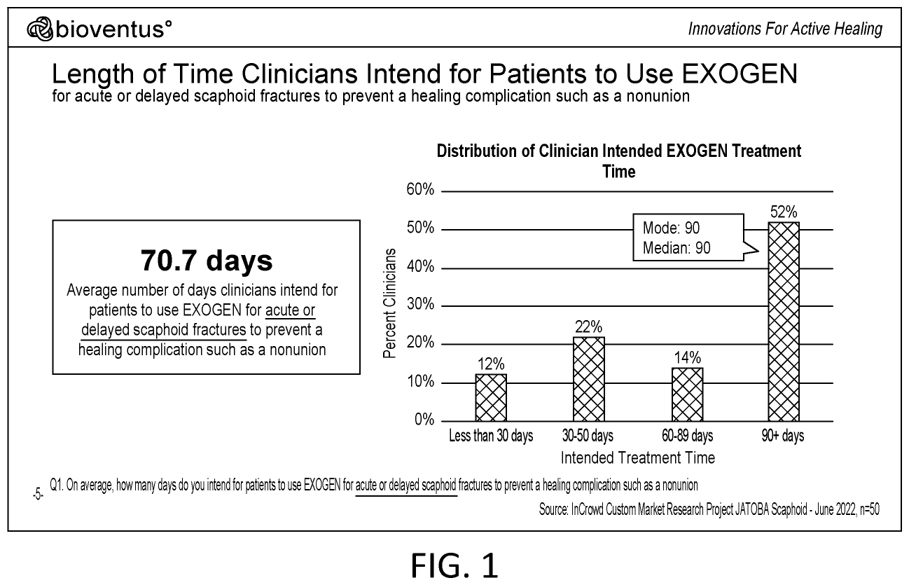

Current standard of care for bone fracture include casting and non-surgical treatments (for example: https://www.aafp.org/pubs/afp/issues/2004/0901/p879.html). Standards of care for low-intensity pulsed ultrasound are limited to treatment at device settings for a prescribed number of minutes for daily use. However, clinicians vary in the amount of days such treatment should continue (i.e. the treatment period over days). Thus, while patients may be compliant with daily usage, adherence to a treatment period suitable for complete healing remains uncertain. Furthermore, patient adherence to a treatment period over a set number of days is challenging. Surveys of clinicians using low-intensity pulsed ultrasound devices for treating bone fractures have found that clinicians continue to use such therapy until complete healing, but generally prescribe use for 70-90 days as seen in FIG. 1.

In one aspect of the method described herein, administration of low-intensity pulsed ultrasound to a patient comprises daily treatment for about 10 minutes to about 30 minutes, about 15 minutes to about 20 minutes, about 10 minutes, about 15 minutes, about 20 minutes, about 25 minutes, or about 30 minutes. In one aspect, the administration of low-intensity pulsed ultrasound to a patient comprises daily treatment for about 20 minutes. In another aspect of the method described herein, administration of low-intensity pulsed ultrasound to a patient comprises once daily, twice daily or three daily treatments. Treatment periods may comprise one or multiple treatments over specified time intervals. In one aspect, the treatment period comprises a daily treatment at the standard device settings for about 1 to about 10 consecutive days, about 1 to about 15 consecutive days, 1 to about 20 consecutive days, 1 to about 25 consecutive days 1 to about 30 consecutive days, 1 to about 45 consecutive days, 1 to about 50 consecutive days, 1 to about 65 consecutive days, 1 to about 75 consecutive days, 1 to about 80 consecutive days, 1 to about 90 consecutive days, 1 to about 100 consecutive days, 1 to about 115 consecutive days, 1 to about 120 consecutive days, 1 to about 125 consecutive days, 1 to about 135 consecutive day, or 1 to about 150 consecutive days.

In yet another aspect of the method described herein, administration of low-intensity pulsed ultrasound to a patient comprises 1 to about 120 continuous treatments (“continuous treatments” referring to consecutive, daily treatments), 1 to about 90 continuous treatments, 1 to about 60 continuous treatments, 1 to about 30 continuous treatments, 1 to about 15 consecutive treatments, about 2 to about 10 consecutive treatments, about 5 to about 20 consecutive treatments, about 10 to about 25 consecutive treatments, about 15 to about 25 consecutive treatments, about 20 to about 30 consecutive treatments, about 25 to about 35 consecutive treatments, about 30 to about 40 consecutive treatments, about 35 to about 45 consecutive treatments, about 40 to about 50 consecutive treatments, about 45 to about 55 consecutive treatments, about 50 to about 60 consecutive treatments, about 55 to about 65 consecutive treatments, about 60 to about 70 consecutive treatments, about 65 to about 75 consecutive treatments, about 70 to about 80 consecutive treatments, about 80 to about 90 consecutive treatments, about 85 to about 95 consecutive treatments, about 90 to about 100 consecutive treatments, about 10 consecutive treatments, about 18 consecutive treatments, about 25 consecutive treatments, about 30 continuous treatments, about 45 continuous treatments, about 60 continuous treatments, about 75 continuous treatments, about 90 continuous treatments, or about 120 continuous treatments. In one aspect of the method described herein, administration of low-intensity pulsed ultrasound to a patient comprises one daily treatment for 20 minutes, over 30 days, over 45 days, over 60 days, over 75 days, over 90 days or over 120 days. In another aspect, administration of the low-intensity pulsed ultrasound to a patient comprises consecutive treatments (consecutive, daily treatments for a defined period of days). In another aspect of the method described herein, the administration of the low-intensity pulsed ultrasound to a patient comprises treatment intervals.

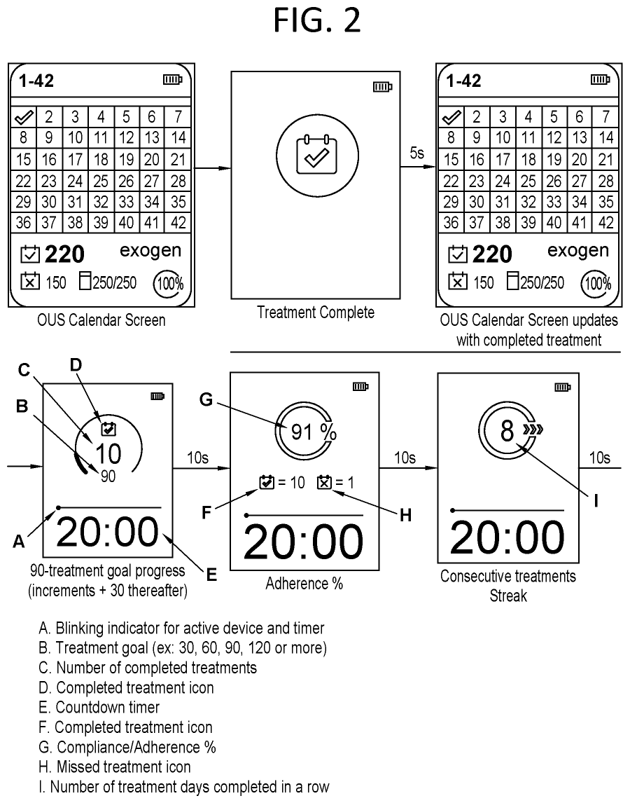

Thus, in another aspect, the method further comprises tracking the patient's treatments. Tracking involves recording the patients treatments, including continuous treatments, and progression towards treatment goal. Tracking thus offers a means for patient compliance and encouragement. Various tools may be used to accomplish recording and tracking. In one aspect, tracking comprises the use of a compliance calendar. A compliance calendar comprises a calendar that the patient uses for reminders for treatment but also to record treatment and thus monitor progression. Compliance calendars may include tracking of treatments performed out of total treatment required to reach goal. In another aspect, the tracking comprises use of a progression toward goal tool. The progression toward goal tool comprises the number of completed treatments, the total treatment goal and a timer to assist the patient in completing their treatment regimen. In another aspect, the tracking comprises use of an adherence tracker that counts the number of full treatments per day since the first treatment. In another aspect, the tracking comprises a continuity tracker which tracks the total number of consecutive treatment days in order to track and encourage a patient to comply with continuous, consecutive, daily treatments. Exemplary tracking tools are described in FIG. 2.

In one aspect, healing of the bone comprises complete healing. Complete healing refers to when a clinician determines that a fracture is healed, typically using X-ray evaluation and clinical evaluation to determine pain, range of motion, strength and other parameters. An evaluation of complete healing may result in a discontinuation of further treatment. In the event complete healing is not achieved, patients may continue treatment for an additional treatment period as determined by a physician. In another aspect, healing of the bone comprises clinically significant healing. Clinically significant healing refers healing having a non-union odds ratio of 1.5, signifying that patients who are not using low-intensity pulsed ultrasound may result in non-union that is 1.5 times more likely that of those using low-intensity pulsed ultrasound.

It will be readily apparent to one of ordinary skill in the relevant arts that suitable modifications and adaptations to the compositions, methods, and applications described herein may be made without departing from the scope of any embodiments or aspects thereof. The compositions and methods provided are exemplary and are not intended to limit the scope of any of the specified embodiments. All of the various embodiments, aspects, and options disclosed herein may be combined in any and all variations or iterations. The scope of the compositions, formulations, methods, and processes described herein include all actual or potential combinations of embodiments, aspects, options, examples, and preferences herein described.

### EXAMPLES

### Example 1

### Low Intensity Pulsed Ultrasound (LIPUS) Therapy to Mitigate the Risk of Non-Union in a Patient at Risk

A nine month study was conducted to demonstrate treatment of a scaphoid fracture with EXOGEN® is effective in reducing the incidence of fracture non-union in patients at risk of scaphoid non-union as compared to the standard of care alone (inclusive of conservative fracture management via casting or surgical repair) using Low Intensity Pulsed Ultrasound (LIPUS). The primary study endpoint was based on the incidence of the scaphoid fracture non-union rate as measured by clinician diagnosis in the form of an International Classification of Diseases (ICD) non-union diagnosis.

### Target Population:

EXOGEN-Treated Cohort: Adult patients (21-60 years old) in the United States (US) at risk for the development of fracture non-union who have been prescribed EXOGEN® as adjunctive treatment for a scaphoid fracture.

### Treatment Regimen:

Patients were given prescriptive therapy with the EXOGEN® Ultrasound Bone Healing System (Model Number 71034400) for a scaphoid fracture as specified by ICD-10-CM code. The EXOGEN® product was used through patient self-administered treatments at home or at work, once daily, for 20 consecutive minutes, as prescribed. Each patient was evaluated and X-rayed by a physician on regular intervals for this data. If a patient was not seen by a physician a patient within the allotted time, the patient was eliminated from the study.

The length of the treatment period window was variable and determined by the treating clinician's assessment of the healing process. In general, clinicians recommend using the EXOGEN® system for treatment of a scaphoid fracture for 90 days to achieve complete healing, complete healing refers to when the clinician determines the fracture was healed. This is typically done through a combination of X-rays and clinical evaluation to determine pain, range of motion, strength, etc.

### Results:

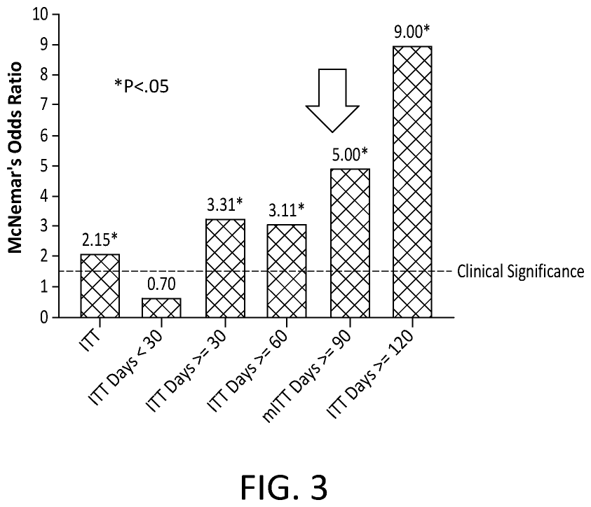

FIG. 3 describes the results as the non-union odds ratio (ITT) for patients using EXOGEN® as compared to the matched cohort. For this study, clinical significance was defined as an odds ratio of 1.5— indicating that the outcome of the control group getting a non-union fracture is 1.5 times that of the group using the EXOGEN® system for treatment. The p-value demonstrates statistical significance.

A correlation between clinical effectiveness and patient compliance with the use of low intensity pulsed ultrasound has been reported (Pounder et al., 2016) with data that eight out of ten clinical trials with patient compliance higher than 72% reported a successful outcome, while two clinical trials reported poor patient compliance, resulting in unsuccessful outcomes. However, compliance was based on daily usage. Thus, while the results indicate a patient may achieve 100% treatment compliance with daily treatments, there is no indication of total number of daily treatments required for complete healing. Thus the results described in FIG. 2 further suggest that complete healing may occur with days of daily treatment than the current standard of care prescribed by physicians.

### Example 2

Future studies similar to the one above will be conducted using patients with tibia fractures.

Although the foregoing disclosure has been described in some detail by way of illustration and example for purposes of clarity of understanding, it will be readily apparent to one of ordinary skill in the art in light of the teachings of this disclosure that certain changes and modifications may be made thereto without departing from the spirit or scope of the appended claims. The following examples are provided by way of illustration only and not by way of limitation. Those skilled in the art will readily recognize a variety of noncritical parameters that could be changed or modified to yield essentially similar results.

1. A method of treating bone fracture in a patient with comorbidities, in need thereof, comprising administering to the patient 30 continuous treatments of a low-intensity pulsed ultrasound to the bone.

2. The method of claim 1 wherein the comorbidities comprise smoking, diabetes, obesity, or cardiovascular disease.

3. The method of claim 1, wherein the bone comprises short bones and long bones.

4. The method of claim 3, wherein the short bone comprises a scaphoid.

5. The method of claim 3, wherein the long bone comprises a tibia.

6. The method of claim 1, further comprising tracking the patient's treatment.

7. The method of claim 6, wherein the tracking comprises using a compliance calendar, a goal progress tracker or a continuity tracker.

8. The method of claim 7, further comprising administration to the patient about 60 to about 120 continuous treatments.

9. The method of claim 8, further comprising administration to the patient about 60 continuous treatments.

10. The method of claim 9, further comprising administration to the patient about 90 continuous treatments.

11. The method of claim 9, further comprising administration to the patient about 120 continuous treatments.

12. The method of claim 1, wherein the method further comprises healing the bone to complete healing.

13. The method of claim 1, wherein the method further comprises healing that is clinically significant.

14. A method of reducing the incidence of non-union fracture of a bone in a patient in need thereof comprising:
a) administering 30 continuous treatments of a low-intensity pulsed ultrasound to a bone;
b) tracking the patient's treatment; and
c) healing the bone to complete healing.

15. The method of claim 14, wherein the bone comprises scaphoid or tibia bone.

16. The method of claim 14, wherein the tracking comprises using a compliance calendar, a goal progress tracker or a continuity tracker.

17. The method of claim 14, further comprising administration of additional 30 continuous treatments.
